(19) United States
(12) Patent Application Publication
(10) Pub. No.: US 2003/0152934 A1
(43) Pub. Date: Aug. 14, 2003

(54) High performance nucleic acid hybridization device and process

(75) Inventors: Yao-Sung Chang, Hsinchu (TW); Yung-Chiang Chung, Hsinchu (TW); Ming-Zheng Shiu, Taichung (TW)

Correspondence Address: Ladas & Parry, 26 West 61st Street, New York, NY, 10023, US

(73) Assignee: INDUSTRIAL TECHNOLOGY RESEARCH INSTITUTE

(21) Appl. No.: 10/073,781

(22) Filed: Feb. 11, 2002

(51) Int. Cl. C12Q 1/68; C12M 1/34

(52) U.S. Cl. 435/6; 435/287.2

(57) ABSTRACT

The invention discloses a device for hybridization reaction between a target molecule in a fluid and a probe, which comprises a microfluidic channel comprising a first portion and a second portion following said first portion; wherein said first portion have an irregular cross section and said second portion has a probe, and a fluid driving element connected the ends of said channel with tubes, wherein said fluid element can move said target molecules back-and-forth for repeatedly passing through said second portion.

### BACKGROUND OF THE INVENTION

1. Field of the Invention

The present invention relates to a device for hybridization reaction between a target molecule in a fluid and a probe, and a process for the hybridization reaction.

2. Description of the Related Prior Art

Molecular biology comprises a wide variety of techniques for the analysis of nucleic acids and proteins. Many of these techniques and procedures form the basis of clinical diagnostic assays and tests. These techniques include nucleic acid hybridization analysis, restriction enzyme analysis, genetic sequence analysis, and the separation and purification of nucleic acids and proteins. For example, nucleic acid hybridizations are now commonly used in genetic research, biomedical research and clinical diagnostics. However, these techniques involve several complex and time-consuming steps. They are normally limited in their applications because of lack of sensitivity, specificity, or reproducibility.

Many apparatuses and methods were developed to improve the efficiency of the hybridization by changing the hybridization conditions. For example, U.S. Pat. No. 5,639,423 is directed to an instrument for in situ chemical reactions in a microfabricated environment. The instrument is especially advantageous for biochemical reactions which require high-precision thermal cycling, particularly DNA-based manipulations such as PCR, since the small dimensions typical of microinstrumentation promote rapid cycling time. U.S. Pat. No. 6,238,910 provides a DNA hybridization apparatus capable of precise thermal and fluid control.

Moreover, some techniques were developed by improving the elements of the apparatus for hybridization assay. U.S. Pat. No. 5,849,486 discloses a system for performing molecular biological diagnosis, analysis and multistep and multiplex reactions utilizing a self-addressable, self-assembling microelectronic system for actively carrying out controlled reactions in microscopic formats. U.S. Pat. No. 6,197,595 provides miniature integrated fluidic systems for carrying out a variety of preparative and analytical operations, as well as methods of operating and using these systems. U.S. Pat. No. 6,255,050 utilizes a force, such as centrifugal force, electrophoretic force, gravitational force vacuum force or pressure, to drive nucleobase-containing sequences in a hybridization reaction that occurs on a partition assembly. U.S. Pat. No. 6,287,850 discloses an agitation system for reversibly directing fluid samples flow back and forth across a nucleic acid array, thereby promoting hybridization between targets in the fluid sample and probes on the nucleic acid array. Further, Liu et al. develops the microfluidic biochemical arrays that integrates massively parallel microfluidic channels with Motorola glass-based microarray biochips (The 14th IEEE international conference on Micro Electro Mechanical System 2001. pp. 439-442. Jan. 21-25, 2001).

However, the above known techniques cannot provide satisfactory efficiency in hybridization and effectively reduce the time needed for hybridization. Consequently, there is still a need to develop a device and method to improve the hybridization assay.

### SUMMARY OF THE INVENTION

An object of the invention is to provide a device for hybridization reaction between a target molecule in a fluid and a probe, which comprises:

a microfluidic channel comprising a first portion and a second portion following said first portion, wherein said first portion has an irregular cross section and said second portion has a probe, and

a fluid driving element connected to the ends of said channel with tubes, wherein said fluid element can move said target molecules back-and-forth for repeatedly passing through said second portion.

Another object of the invention is to provide a process for increasing hybridization reaction between a target molecule and a probe, which comprises the following steps:

(a) providing a microfluidic channel comprising a first portion and a second portion following said first portion, wherein said first portion has an irregular cross section and said second portion has a first probe and second or more probes wherein said first probe specifically binds to said target molecule;

(b) introducing a fluid containing said target molecule into the microfluidic channel of the device for hybridization reaction of the invention;

(c) driving said fluid to flow back and forth so that said target molecule can repeatedly pass through said second portion, whereby said target molecules non-specifically binding to the second or more probes are removed and the target molecules binding to said first probe are retained.

### BRIEF DESCRIPTION OF THE DRAWINGS

FIG. 1 shows the examples of the shape of the irregular cross section of microfluidic channel of the device for hybridization reaction.

FIG. 2 illustrates the device of the invention.

FIG. 3 shows the microfluidic channels with different shapes (Device I: circle and Device II: straight) of irregular cross sections.

FIG. 4 shows that the hybridization efficiency of the target DNA driven by the micropump is better than that of the incubated target DNA (control), regardless of the shape of the irregular cross section.

FIG. 5 shows that the hybridization efficiency of the circle irregular section.

FIG. 6 shows the microfluidic channels with different sizes of cross sections.

FIG. 7 shows that the hybridization efficiency of the target DNA driven by the micropump is better than that of the incubated target DNA (control), no matter in Device III or IV.

FIG. 8 shows that the hybridization signal after 30 minutes in the slow region (large cross section) of Device III is 1.5 times of that after 4 hours of the control.

FIG. 9 shows that the hybridization signal after 30 minutes in the slow region (large cross section) of Device IV is 2.7 times of that after 4 hours of the control, i.e. 6.1 times of the hybridization signal after 30 minutes of the control.

### DETAILED DESCRIPTION OF THE INVENTION

The present invention utilizes the microfluidic channel capable of producing shear stress and the forward and backward movement of the fluid to increase the hybridization efficiency between the target molecules in the fluid and the probes and reduce the time needed for hybridization.

An object of the invention is to provide a device for hybridization reaction between a target molecule in a fluid and a probe, which comprises:

a microfluidic channel comprising a first portion and a second portion following said first portion, wherein said first portion has an irregular cross section and said second portion has a probe, and

a fluid driving element connected to the ends of said channel with tubes, wherein said fluid element can move said target molecules back-and-forth for repeatedly passing through said second portion.

According to the invention, the probe is a surface-immobilized molecule that is recognized by a particular target and is sometimes referred to as a ligand. Examples of probes that can be investigated by this invention include, but are not restricted to, protagonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides or nucleic acids, oligosaccharides, proteins, and monoclonal antibodies.

According to the invention, a target molecule is that having an affinity to a given probe and is sometimes referred to as a receptor. Target molecules may be naturally-occurring or artificial molecules. Also, they can be employed in their unaltered state or as aggregates with other species. Target molecules may be attached, covalently or noncovalently, to a binding member, either directly or via a specific binding substance. Examples of target molecules which can be employed by this invention include, but are not limited to, antibodies, cell membrane receptors, monoclonal antibodies and antisera reactive with specific antigenic determinants (such as on viruses, cells or other materials), drugs, oligonucleotides or nucleic acids, peptides, cofactors, lectins, sugars, polysaccharides, cells, cellular membranes, and organelles. Preferably, the target molecule of the invention is nucleic acid, peptide or peptide ribonucleic acid. More preferably, the target molecule of the invention is DNA or RNA. Even more preferably, the target molecule is single-stranded nucleic acid or double-stranded nucleic acid.

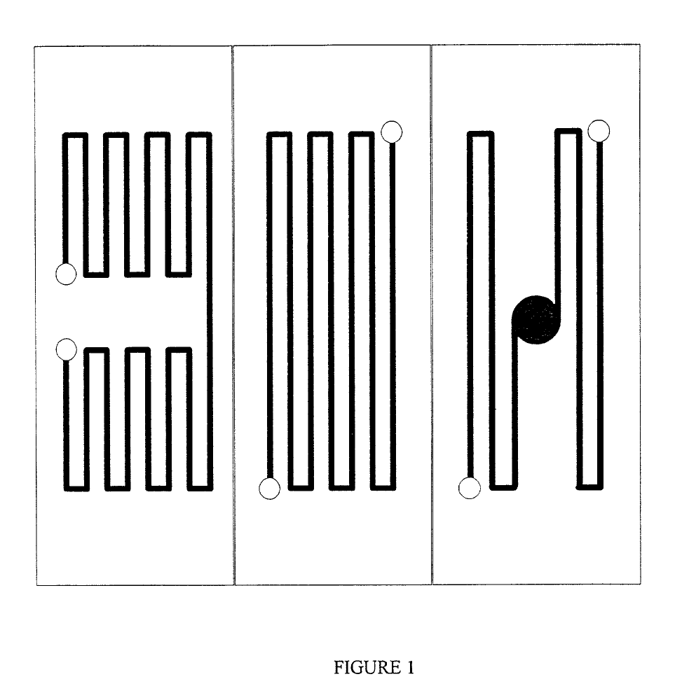

According to the invention, the microfluidic channel of the device comprises a first portion and a second portion following said first portion. According to the invention, the first portion has an irregular cross section. The irregular cross section is produced by irregularly changing the size of the cross section of said first portion of said channel. Examples of the shape of the first portion are shown in FIG. 1. Most molecules of the single-stranded nucleic acids may form a coiled conformation due to the formation of the intra-molecular hydrogen bond. In view of such conformation, the area for conducting hybridization reaction is located inside the conformation of molecule so that the hybridization reaction is not complete. In the past, only about 8% of nucleic acid molecules can be completely reacted. According to the invention, the first portion can produce shear stress capable of stretching the nucleic acid molecule to a linear form that is in favor of the hybridization reaction. In addition, the double-stranded nucleic acid also can be used in the device of the invention. The shear stress produced by the first portion of the invention can denature the double-stranded nucleic acid to produce the single-stranded nucleic acid. Similarly, the reaction sites of protein molecules may be located inside of the three-dimensional structure. The shear stress can damage the three-dimensional structure of the protein so that the reaction sites are exposed outside for easily carrying out the hybridization reaction. According to the invention, the inner surface of the microfluidic channel is rough or has recess slots.

According to the invention, the device comprises a fluid driving element connected the ends of said channel with tubes. Preferably, the fluid driving element is a gas driving micropump, mechanical micropump or electrokinetic micropump. More preferably, the mechanical micropump is selected from the group consisting of electrostatic micropump, magnetically driven micropump, diffuser micropump. Even more preferably, the electrokinetic micropump is selected from the group consisting of electrohydrodynamics micropump and electrophoretic micropump and electroosmotic micropump.

According to the invention, the device further comprises a means for providing energy to said target molecules. Preferably, the means is a heater such as a thermal cycler. The energy can increase the number of collisions between the target molecules and the probes. The hybridization efficiency can be thus increased.

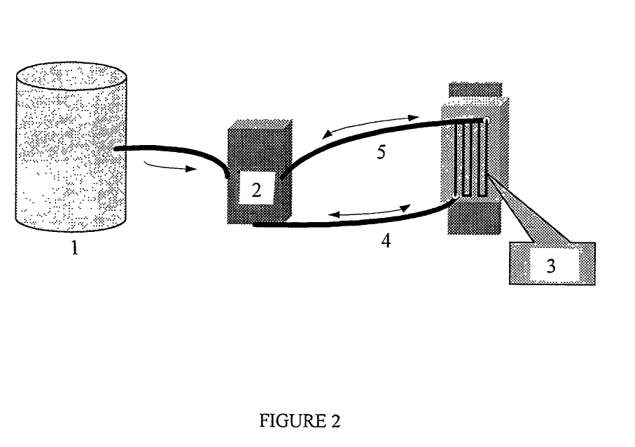

One preferred embodiment of the invention is provided to illustrate the device for hybridization reaction between a target molecule in a fluid and a probe (see FIG. 2). A micropump 1 drives the fluid to flow to a valve 2. The fluid flows into the microfluidic channel 3 through tube 4 and a hybridization reaction is carried out in channel 3. The resulting fluid flows out of channel 3 through a tube 5.

According to the invention, any known techniques (such as micromolding, etching and bonding approaches) can be used to fabricate the device for hybridization reaction of the invention such as the method described in “The 14th IEEE international conference on Micro Electro Mechanical System 2001. pp. 439-442. Jan. 21-25, 2001.” Preferably, the device of the invention can be used in removing the target molecules nonspecifically binding to the probes.

Another object of the invention is to provide a process for removing non-specifically binding target molecules in hybridization reaction between a target molecule and a probe, which comprises the following steps:

(a) providing a microfluidic channel comprising a first portion and a second portion following said first portion; wherein said first portion has an irregular cross section and said second portion has a first probe and second or more probes wherein said first probe specifically binds to said target molecule;

(b) introducing a fluid containing said target molecule into the microfluidic channel of the device for hybridization reaction of the invention;

(c) driving said fluid to flow back and forth so that said target molecule can repeatedly pass through said second portion, whereby said target molecules non-specifically binding to the second or more probes are removed and the target molecules binding to said first probe are retained.

According to the invention, the microfluidic channel used in the process for removing non-specifically binding target molecules comprises a first portion having an irregular cross section and a second portion having a first probe and second or more probes wherein said first probe specific binds to said target molecule. The target molecules nonspecifically binding to the other probes can be removed by driving the fluid repeatedly to pass through said second portion.

According to the invention, the device and process of the invention can reduce the time needed for hybridization reaction and increase the hybridization efficiency. The present invention provides commercially feasible devices for conducting hybridization reaction. It is to be understood that the above description is intended to be illustrative but not restrictive. Many embodiments will be apparent to those skilled in the art upon reviewing the above description.

### EXAMPLES

### Example 1

Hybridization Reaction

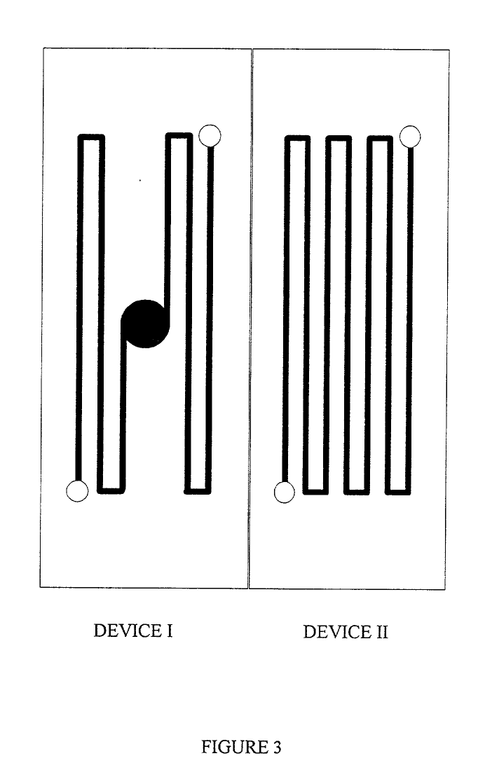

The hybridization efficiencies of microfluidic channels with different shapes (FIG. 3, Device I: circle; Device II: straight) are compared in this example.

The four probes, Sp5 (0.5 μM), Alo3 (5 μM), Alo1 (5 μM) and P3 (5 μM), are respectively spotted onto the chip with a pipetman (Immobilization buffer: 2×SSC; Chip: sol-gel developed by Industrial Technology Research Institute, Volume of each spot: 200 nl) and allowed to react at 37° C. for 4 hours. The chip is then sonicated in 0.5% SDS, washed twice with deionized water for 1 minute, and air-dried in a DNA hybridization oven (Hybrid Inc.).

A specific mould mask with a microfluidic channel thereon comprising a circle or straight first portion is tightly combined with the above chip with the specific nucleic acid probes fixed thereon by a strong spring clip. The microfluidic channel on the mould mask should accurately cover the nucleic acid probes fixed on the chip and so the nucleic acid probes are exposed in the microfluidic channel.

The target DNA, Cy5O3 (1 μM; a single-stranded 25 bp sequence complementary to that of Alo3, with its 5′ end labeled by Cy5 fluorescence for detection), is denatured. The fluid containing 10 μl of target DNA, 20 μl of deionized water and 30 μl of 2× hybridization buffer is introduced into the microfluidic channel. An additional energy is provided to increase the hybridization. The target DNA is driven back and forth by a micropump to perform hybridization (40° C.; 1 hour). Separately, the target DNA is introduced into another microfludic channel and incubated at 40° C. for 1 hour to perform hybridization (as the control). After the hybridization, the chip is detected the fluorescence with a scanner (ScanArray 4000, General Scanning Inc.).

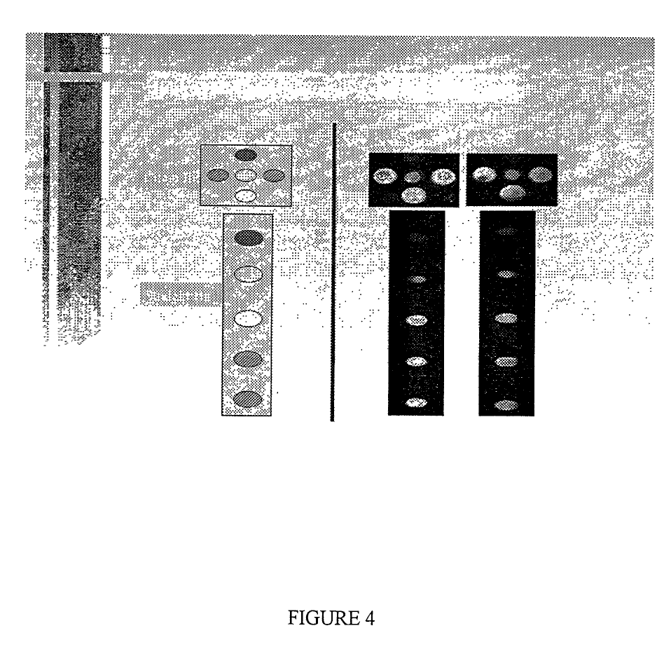

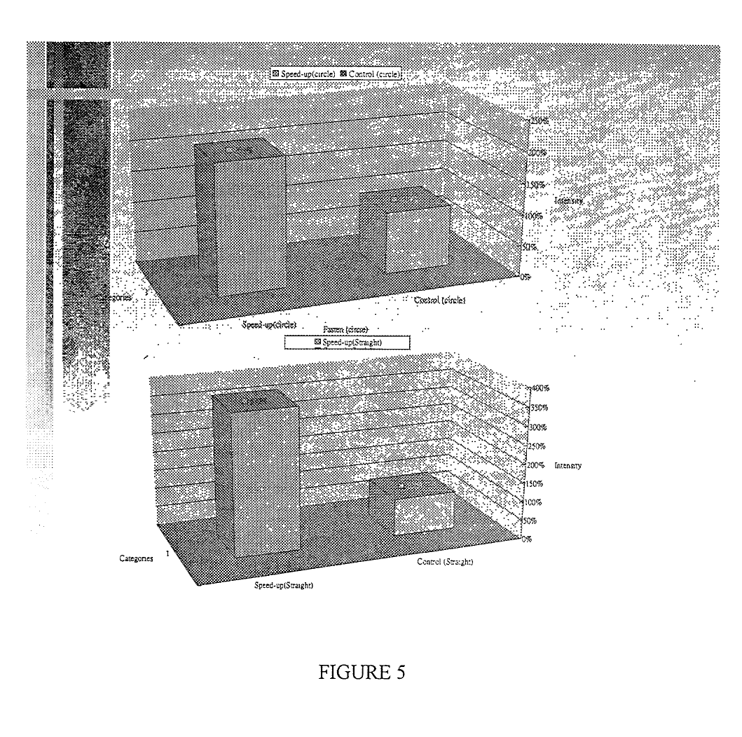

The result is shown in FIGS. 4 and 5. FIG. 4 shows that the hybridization efficiency of the target DNA driven by the micropump is better than that of the incubated target DNA (control), regardless of the shape of the first portion. FIG. 5 shows that the hybridization efficiency of the straight first portion (the signal is about 3.8 times of that of the control) is better than that of the circle one (the signal is about 2.1 times of that of the control).

### Example 2

Hybridization Reaction

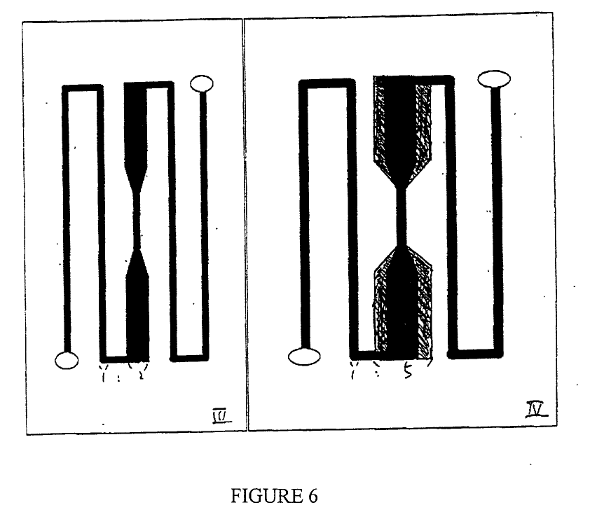

The hybridization efficiencies of microfluidic channels with different sizes of cross sections (FIG. 6) are compared in this example.

The five probes, Sp5 (0.5 μM), No2 probe (5 μM), No3 probe (5 μM), No4 probe (5 μM) and No5 probe (5 μM), are respectively dotted onto the chip with a pipetman (Immobilization buffer: 2×SSC; Chip: sol-gel developed by Industrial Technology Research Institute; Volume of each spot: 200 nl) and allowed to react at 37° C. for 4 hours. The chip is then sonicated in 0.5% SDS, washed twice with deionized water for 1 minute, and air-dried in a DNA hybridization oven (Hybrid Inc.).

Two straight microfluidic channels, with 2:1 (Device III) and 5:1 (Device IV) cross sections, were produced as described in Example 1 and are used in the following hybridization test.

The target DNA (a single-stranded 1 kb sequence complementary to that of No5, with its 5′ end labeled by Cy5 fluorescence for detection) is denatured. The fluid containing 10 μl of target DNA, 20 μl of deionized water and 30 μl of 2× hybridization buffer is introduced into the microfluidic channel. The target is driven back and forth by a micropump to perform hybridization (40° C.; 30 minutes). Separately, the same target DNA is introduced into another microfluidic channel and incubated at 40° C. for 30 minutes to perform hybridization (as the control). After the hybridization, the chip is detected the fluorescence with a scanner (ScanArray 4000, General Scanning Inc.).

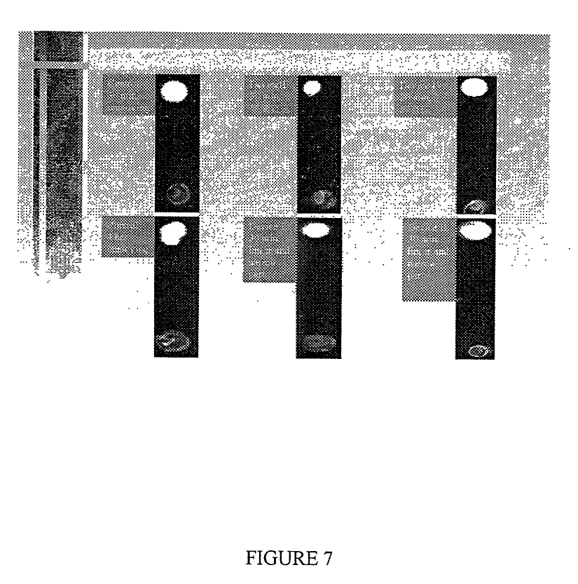

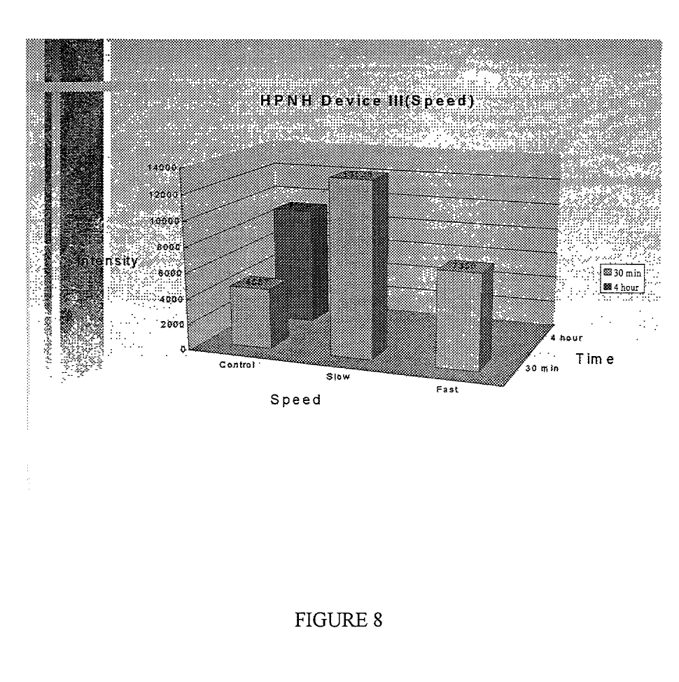

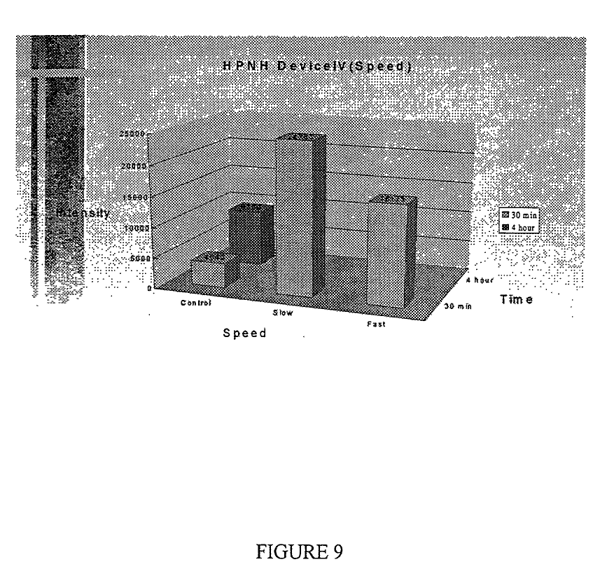

The result is shown in FIGS. 7 to 9. FIG. 7 shows that the hybridization efficiency of the target DNA driven by the micropump is better than that of the incubated target DNA (control), no matter in Device III or IV. FIG. 8 shows that the hybridization signal after 30 minutes in the slow region (large cross section) of Device III is 1.5 times of that after 4 hours of the control. FIG. 9 shows that the hybridization signal after 30 minutes in the slow region (large cross section) of Device IV is 2.7 times of that after 4 hours of the control, i.e. 6.1 times of the hybridization signal after 30 minutes of the control. These results imply that the magnitude of the hybridization signal is not only influenced by the kinetic energy provided to drive the target DNA, but also by the design of the microfluidic channels.

While the invention has been particularly shown and described with the reference to the preferred embodiment thereof, it will be understood by those skilled in the art that various changes in form and details may be made without departing from the spirit and scope of the invention.

We claim:

1. A device for hybridization reaction between a target molecule in a fluid and a probe, which comprises:
a microfluidic channel comprising a first portion and a second portion following said first portion, wherein said first portion has an irregular cross section and said second portion has a probe, and
a fluid driving element connected the ends of said channel with tubes, wherein said fluid element can move said target molecules back-and-forth for repeatedly passing through said second portion.

2. The device of claim 1, wherein said irregular cross section is produced by irregularly changing the size of the cross section of said first portion of said channel.

3. The device of claim 1, wherein the inner surface of said microfluidic channel is rough or has recess slots.

4. The device of claim 1, wherein said probe is nucleic acid, peptide or peptide nucleic acid.

5. The device of claim 4, wherein said nucleic acid is DNA or RNA.

6. The device of claim 4, wherein said nucleic acid is single-stranded nucleic acid or double-stranded nucleic acid.

7. The device of claim 1, which further comprises a means for providing energy to said target molecules.

8. The device of claim 1, which can be used in removing the target molecules non-specific binding to said probes.

9. A process for increasing hybridization reaction between a target molecule and a probe, comprising the following steps:
(a) providing a microfluidic channel comprising a first portion and a second portion following said first portion, wherein said first portion has an irregular cross section and said second portion has a first probe and second or more probes wherein said first probe specific binds to said target molecule;
(b) introducing a fluid containing said target molecule into the microfluidic channel of the device for hybridization reaction of the invention;
(c) driving said fluid to flow back and forth so that said target molecule can repeatedly pass through said second portion, whereby said target molecules non-specific binding to the second or more probes are removed and the target molecules binding to first probe are retained.

10. The process of claim 9, wherein said probe is nucleic acid, is peptide or peptide nucleic acid.

11. The process of claim 10, wherein said nucleic acid is DNA or RNA.

12. The process of claim 10, wherein said nucleic acid is single-stranded nucleic acid or double-stranded nucleic acid.

13. The process of claim 9, wherein the surface of said channel is rough.

14. The process of claim 9, wherein said irregular cross section is produced by irregularly changing the size of the cross section of said first portion of said channel.

15. The device of claim 9, which further comprises a step for providing energy to said target molecules.
